(19) United States
(12) Patent Application Publication
(10) Pub. No.: US 2026/0248624 A1
(43) Pub. Date: Aug. 27, 2026

(54) IMPLANTABLE MEDICAL DEVICE WITH A FLUIDIC MANIFOLD HAVING A STRUCTURAL LINER AND A CIRCUIT BOARD

(71) Applicant: Boston Scientific Scimed, Inc., Maple Grove, MN (US)

(72) Inventors: John Anders Bostrom, Saint Paul, MN (US); Trey Henry Achterhoff, Arden Hills, MN (US); Derek John Boettger, Woodbury, MN (US); Matthew William Couri, Plymouth, MN (US); Henry Koon, Saint Paul, MN (US); Paul John Gindele, Buffalo, MN (US)

(21) Appl. No.: 19/466,911

(22) Filed: Feb. 2, 2026

Related U.S. Application Data
(60) Provisional application No. 63/754,091, filed on Feb. 5, 2025.

(51) Int. Cl. A61F 2/48; A61F 2/00; A61F 2/26

(52) U.S. Cl. CPC A61F 2/482; A61F 2/004; A61F 2/26; A61F 2240/001

(57) ABSTRACT

According to an aspect, an implantable medical device may include an inflatable member, a fluid reservoir, and an electronic pump device configured to transfer fluid between the inflatable member and the fluid reservoir. The electronic pump device includes a housing. The housing includes a fluidic manifold including a plurality of fluidic components. The fluidic manifold includes an inside edge and a shelf that extends from the inside edge. The fluidic manifold includes a circuit substrate contacting the shelf, and a structural liner connecting the inside edge and the shelf.

### CROSS-REFERENCE TO RELATED APPLICATION

This application claims priority to U.S. Provisional Patent Application No. 63/754,091, filed on Feb. 5, 2025, entitled “IMPLANTABLE MEDICAL DEVICE WITH A FLUIDIC MANIFOLD HAVING A STRUCTURAL LINER AND A CIRCUIT BOARD”, the disclosure of which is incorporated by reference herein in its entirety.

### TECHNICAL FIELD

This disclosure relates generally to an implantable medical device with an electronic pump device having a fluidic manifold with a structural liner and a circuit board.

### BACKGROUND

Some inflatable medical devices have a manual pump, which, when operated by a user, causes a transfer of fluid between a fluid reservoir and an inflatable member. In some examples, an inflatable medical device may include an electronic pump. External loads on the surface(s) of the electronic pump assembly may cause displacement of the individual components of the electronic pump assembly, which may affect the operation of the inflatable medical device.

### SUMMARY

In some aspects, the techniques described herein relate to an implantable medical device including: an inflatable member; a fluid reservoir; and an electronic pump device configured to transfer fluid between the inflatable member and the fluid reservoir, the electronic pump device including a housing, the housing including: a fluidic manifold including a plurality of fluidic components, the fluidic manifold including an inside edge and a shelf that extends from the inside edge; a circuit substrate contacting the shelf; and a structural liner connecting the inside edge and the shelf.

In some aspects, the techniques described herein relate to an electronic pump device for an inflatable medical device, the electronic pump device including: a fluidic manifold including a plurality of fluidic components, the fluidic manifold including an inside edge and a shelf that extends from the inside edge; a circuit substrate contacting the shelf; and a structural liner connecting the inside edge and the shelf.

In some aspects, the techniques described herein relate to a method including: positioning a circuit substrate on a shelf of a fluidic manifold of an electronic pump device of an implantable medical device; positioning a structural liner on the fluidic manifold; and coupling the circuit substrate and the structural liner to the shelf using a coupling member.

In some aspects, the techniques described herein relate to an implantable medical device including: an inflatable member; a fluid reservoir; and an electronic pump device configured to transfer fluid between the inflatable member and the fluid reservoir, the electronic pump device including a housing, the housing including: a fluidic manifold including a plurality of fluidic components, the fluidic manifold including an inside edge and a shelf that extends from the inside edge; a circuit substrate contacting the shelf; and a structural liner connecting the inside edge and the shelf.

In some aspects, the techniques described herein relate to a method including: positioning a circuit substrate on a shelf of a fluidic manifold of an electronic pump device of an implantable medical device; positioning a structural liner on the fluidic manifold; and coupling the circuit substrate and the structural liner to the shelf using a coupling member.

In some aspects, the techniques described herein relate to an electronic pump device for an inflatable medical device, the electronic pump device including: a fluidic manifold including a plurality of fluidic components, the fluidic manifold including an inside edge and a shelf that extends from the inside edge; a circuit substrate contacting the shelf; and a structural liner connecting the inside edge and the shelf.

### BRIEF DESCRIPTION OF THE DRAWINGS

FIG. 1A illustrates an implantable medical device with an electronic pump device having a fluidic manifold, a liner, and a circuit board according to an aspect.

FIG. 1B illustrates an example of a fluidic manifold with a liner and a circuit board according to an aspect.

FIG. 1C illustrates an example of a fluidic manifold according to an aspect.

FIG. 1D illustrates an example of a fluidic manifold with a liner according to an aspect.

FIG. 1E illustrates an example of a fluidic manifold with a liner according to another aspect.

FIG. 1F illustrates an example of a fluidic manifold with a circuit board according to an aspect.

FIG. 1G illustrates an example of a protrusion extending from a shelf of a fluidic manifold according to an aspect.

FIG. 2 illustrates an exploded view of a housing of an electronic pump device according to an aspect.

FIG. 3A illustrates an example of a liner coupled to a protrusion of a fluidic manifold based on an interference fit according to an aspect.

FIG. 3B illustrates a cross-sectional view of a liner coupled to a protrusion of a fluidic manifold according to aspect.

FIG. 4A illustrates an example of a clip coupled to a post for coupling a liner and a circuit board to a shelf of a fluidic manifold according to an aspect.

FIG. 4B illustrates a cross-sectional view of a fluidic manifold with a circuit board, liner, and clip according to an aspect.

FIG. 5 illustrates an example of a liner with a protrusion for interacting with a hole on a shelf of a fluidic manifold according to an aspect.

FIGS. 6A and 6B illustrate an example of using a heat stake or material reflow retainment for coupling a liner and a circuit board to a shelf of a fluidic manifold according to an aspect.

FIGS. 7A to 7D illustrate an example of using rivets for coupling a liner and a circuit board to a shelf of a fluidic manifold according to an aspect.

FIG. 8 illustrates an example of using fasteners for coupling a liner and a circuit board to a shelf of a fluidic manifold according to an aspect

FIG. 9 illustrates an example of using a snap fit feature on a liner according to an aspect.

FIG. 10 illustrates an example of using a snap fit feature on a liner according to another aspect.

FIG. 11 illustrates a perspective of an inflatable penile prosthesis according to an aspect.

FIG. 12 illustrates an example of an artificial urinary sphincter device according to an aspect.

FIG. 13 illustrates a flowchart depicting example operations of coupling a liner and a circuit substrate to a shelf of a fluidic manifold of an electronic pump device according to an aspect.

### DETAILED DESCRIPTION

This disclosure relates to an implantable medical device with an electronic pump device configured to automatically transfer fluid between a fluid reservoir and an inflatable member. In some examples, the implantable medical device includes a penile prosthesis with one or more inflatable cylinders. In some examples, the implantable medical device includes a urinary control device with an inflatable cuff. However, the implantable medical device may include other types of medical devices such as a stimulation device or a neuromodulation device. The electronic pump device includes a housing with a fluidic manifold having pumps (e.g., piezo-electric pumps) and valve(s)) to facilitate the transfer of fluid between the fluid reservoir and the inflatable member. The fluidic manifold includes a frame with a shelf that retains and is coupled to a circuit substrate and a structural liner. The shelf may include a planar surface on the inside edge of the fluidic manifold. The shelf may support and align the circuit substrate and structural liner. The structural liner may increase the integrity and reliability of the electronic pump device by minimizing the displacement of components included within the electronic pump device.

The circuit substrate may contact the manifold’s shelf. In some examples, the structural liner includes one or more portions that contact the circuit substrate. In some examples, one or more circuit substrate portions are located between the structural liner and shelf. The electronic pump device includes coupling member(s) that couple the structural liner (and the circuit substrate) to the fluidic manifold (e.g., in some examples, to the shelf of the fluidic manifold). In some examples, a coupling member includes one or more male features (e.g., protrusions, posts, fasteners, rivets, etc.) and one or more female features (e.g., holes, slots, opening, etc.), where the male feature can be defined on the fluidic manifold and the female feature can be defined on the structural liner or the male feature can be defined on the structural liner and the female feature can be defined on the fluidic manifold. In some examples, the coupling member includes one or more protrusions (e.g., posts) (e.g., a solid or grooved posts) that extend from the manifold’s shelf, and the protrusions extends through one or more first holes on the circuit substrate and one or more second holes on the structural liner. In some examples, the structural liner includes one or more male features (e.g., posts) and the shelf includes one or more female features (e.g., holes). In some examples, a protrusion includes a post that is integral to and extends from the shelf. In some examples, the post is a cylindrical post.

In some examples, the coupling member(s) may enable a press-fit or an interference fit. For example, the structural liner may be coupled to the fluidic manifold based on a press-fit or interference fit. The fluidic manifold may have male features that interface with female features of the structural liner (or vice versa), thereby providing an interference fit to minimize or prevent movement. In some examples, the size (e.g., the diameter) of the second holes on the structural liner are less than the size (e.g., the diameter) of the protrusions. In some examples, an assembly operator may forcibly press fit the structural liner onto the manifold posts in one, two, or more locations, thereby securing the circuit substrate and minimizing (e.g., preventing) movement of the device’s component relative to one another.

In some examples, the coupling mechanism includes one or more clips (e.g., e-style clips) that can be pushed on the male features (e.g., protrusions), thereby mechanically locking the structural liner and the circuit substrate to the manifold’s shelf. For example, a clip can be pushed onto a protrusion to hold the structural liner and the circuit substrate. In some examples, the structural liner is secured to the male features (e.g., posts) by using a heat stake or material reflow (e.g., material on the manifold is melted to lock down the structural liner and the circuit substrate).

In some examples, the coupling member includes one or more rivets. In some examples, the rivets include push-in rivet style fasteners. In some examples, the rivets are used to connect through holes, thereby securing (e.g., locking) both the circuit substrate and the structural liner. In some examples, the coupling member includes one or more fasteners. In some examples, the fasteners include screws, and the holes are threaded holes. In some examples, a screw is loaded from the top side of the structural liner threading into a pre-drilled hole on the fluidic manifold. In some examples, the coupling member includes a snap fit with an undercut. In some examples, the fluidic manifold has an undercut that engages with a snap-fit feature on the structural liner to secure the structural liner to the fluidic manifold. In some examples, the coupling member includes an adhesive material disposed on the protrusion, thereby securing the structural liner and the circuit substrate. In some examples, the coupling member includes a ring member coupled to the male feature (e.g., the protrusion), where a portion of the circuit substrate and a portion of the structural liner are located between the ring member and the shelf. In some examples, the coupling member includes injection molding.

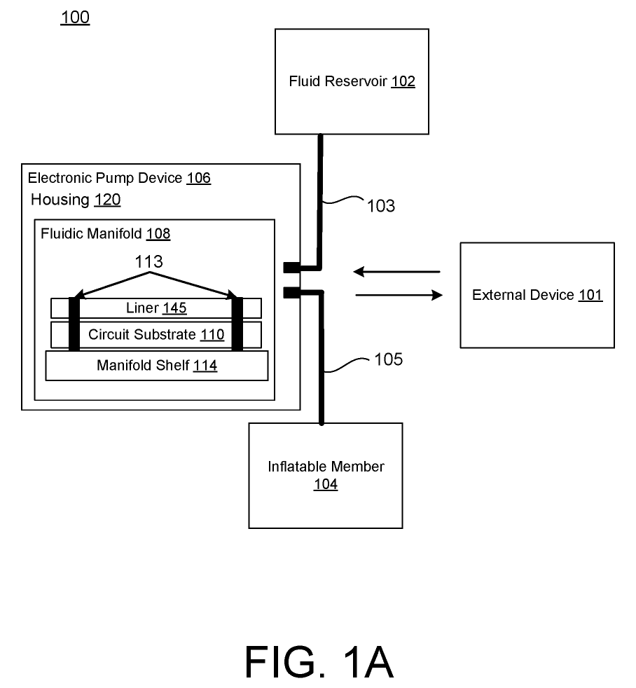

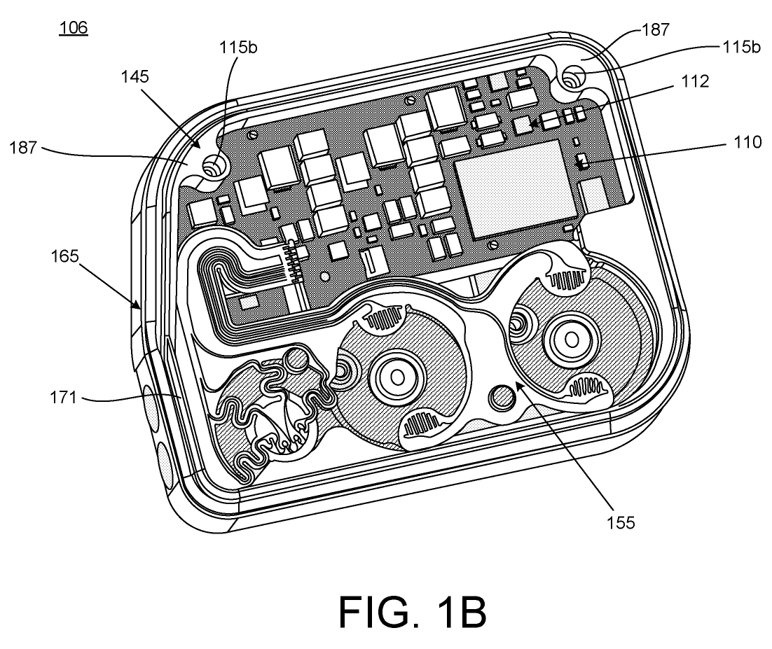

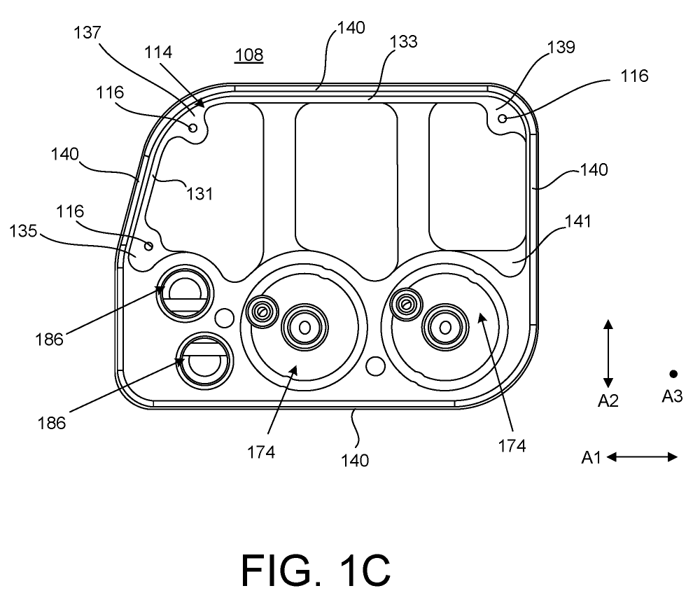

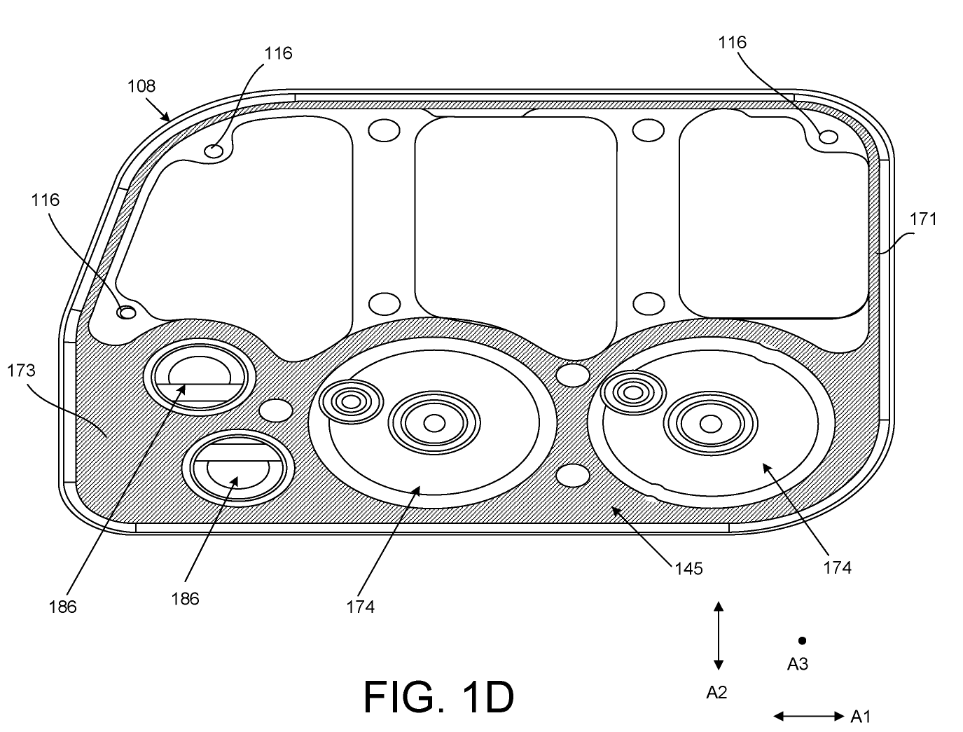

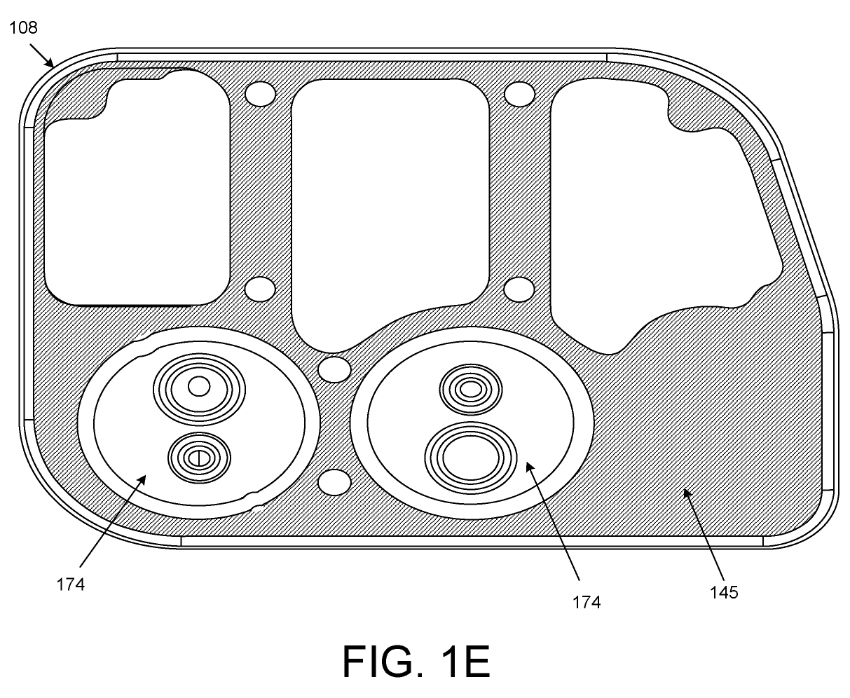

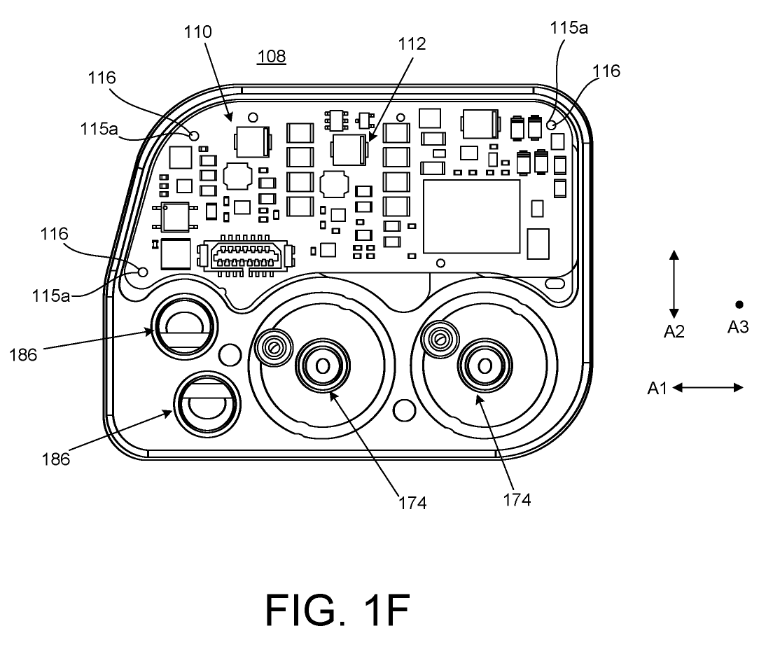

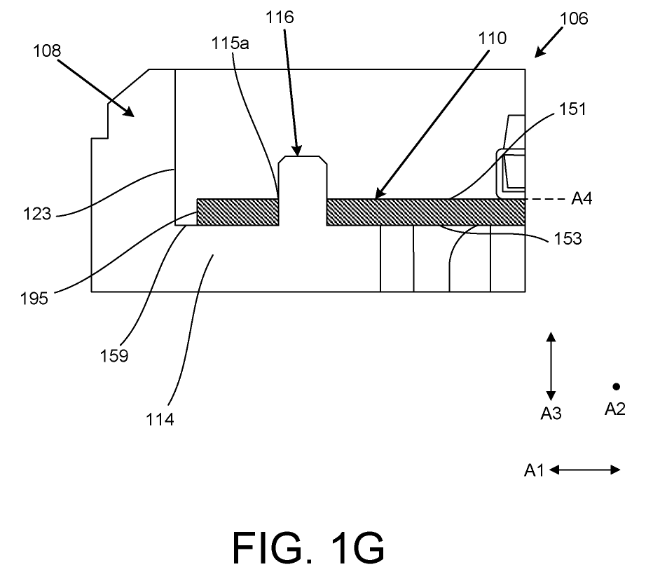

FIGS. 1A-1G illustrate an implantable medical device 100 according to an aspect. In some examples, the implantable medical device 100 is an artificial urinary sphincter device. In some examples, the implantable medical device includes a urinary control device with an inflatable cuff. In some examples, the implantable medical device 100 is an inflatable penile prosthesis. However, the implantable medical device 100 may include any type of medical device that transfers fluid between components of the implantable medical device 100 such as respiratory devices, cardiovascular devices, gastrointestinal devices, ophthalmic devices, drug delivery devices, and/or diagnostic devices. In some examples, the implantable medical device 100 may be a stimulation device. In some examples, the implantable medical device 100 may be a neuromodulation device.

The implantable medical device 100 includes an electronic pump device 106. The electronic pump device 106 includes a housing 120 with a fluidic manifold 108. The fluidic manifold 108 includes fluidic components 174 (e.g., pump(s) and valve(s)) to facilitate the transfer of fluid between a fluid reservoir 102 and an inflatable member 104. In some examples, the fluidic manifold 108 can be referred to as a frame or an internal frame of the housing 120.

The housing 120 includes a circuit substrate 110 coupled to the fluidic manifold 108. The circuit substrate 110 includes circuit components 112. The housing 120 includes a structural liner 145 coupled to the fluidic manifold 108. The fluidic manifold 108 includes a shelf 114 that contacts and is coupled to the circuit substrate 110 and the structural liner 145. The structural liner 145 may increase the integrity and reliability of the electronic pump device 106 by minimizing the displacement of components (e.g., the valve(s), pump(s), pressure sensors, etc.) included within the electronic pump device 106.

FIG. 1B illustrates a perspective of the electronic pump device 106 having a structural liner 145 and a circuit substrate 110 coupled to the fluidic manifold 108. FIG. 1C illustrates an example of the fluidic manifold 108 without the structural liner 145 and the circuit substrate 110. FIG. 1D illustrates one side (e.g., front or back side) of a portion of the structural liner 145 that contacts a surface area of the fluidic manifold in directions A1 and A2. FIG. 1E illustrates another side (e.g., back side or front side) of the portion of the structural liner 145 that contacts a surface area of the fluidic manifold in the directions A1 and A2. FIG. 1F illustrates an example of the fluidic manifold 08 with the circuit substrate 110. FIG. 1G illustrates a cross-sectional view of the fluidic manifold 108 with the circuit substrate 110.

In some examples, as shown in FIG. 1B, the housing 120 includes a flex circuit 155 coupled to the fluidic manifold 108. The flex circuit 155 may include a flexible substrate. The flex circuit 155 may be disposed on and contact portions of the circuit substrate 110, one or more pressure sensors 186, and one or more fluidic components 174. In some examples, the flex circuit 155 may contact one or more portions of the inside edge 123. In some examples, the housing 120 includes an outer liner 165 coupled to an outside edge of the fluidic manifold 108.

Referring to FIG. 1A, the electronic pump device 106 includes one or more coupling members 113 that couples the circuit substrate 110 and the structural liner 145 to the fluidic manifold 108 (e.g., to the shelf 114 of the fluidic manifold 108). In some examples, the coupling members 113 includes one or more male features (e.g., protrusions, posts, fasteners, rivets, etc.) and one or more female features (e.g., holes, slots, openings, etc.). The male feature may be defined on the fluidic manifold 108 (e.g., the shelf 114 of the fluidic manifold 108) and the female features may be defined on the structural liner 145. In some examples, the female feature may be defined on the fluidic manifold 108 (e.g., the shelf 114 of the fluidic manifold 108) and the male features may be defined on the structural liner 145.

In some examples, as shown in FIGS. 1B to 1G, the coupling members 113 include protrusions 116 (e.g., posts, cylindrical posts, grooved posts, extension members, etc.) that extend from the shelf 114, and the protrusions 116 extend through holes 115a on the circuit substrate 110 and into (or, in some examples, through) holes 115b on the structural liner 145. In some examples, the interaction between the protrusions 116 and the holes 115b form a press-fit coupling mechanism. Although some examples use a press-fit coupling mechanism, the structural liner 145, the circuit substrate 110, and the fluidic manifold 108 may be coupled to each other based on other types of coupling mechanisms.

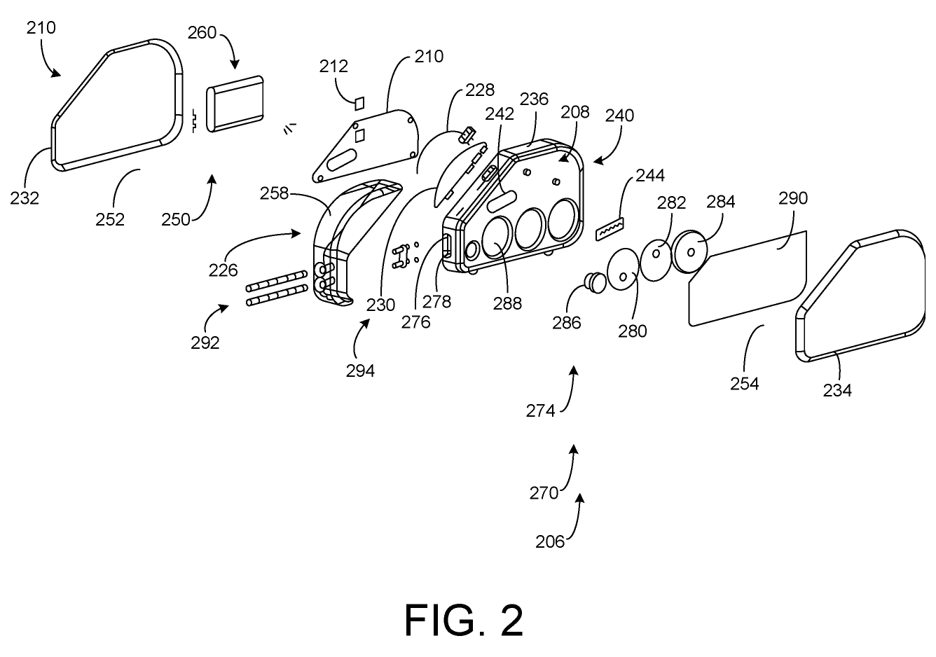

The fluidic manifold 108 may attach fluid transfer and pressure regulating components. For example, the fluidic manifold 108 may include fluidic components 174 (e.g., pump(s) and valve(s)) and pressure sensors 186. The fluidic manifold 108 may also attach a circuit substrate 110 with circuit components 112 (e.g., electronic components). In some examples, the circuit substrate 110 is a printed circuit board (PCB). The fluidic manifold 108 includes a frame 140 with an inside edge 123 (e.g., that extends in a direction A3). The frame 140 may define a peripheral wall (e.g., a peripheral wall 236 of FIG. 2), which is a portion of the outer surface of the electronic pump device 106. As shown in FIG. 1G, the fluidic manifold 108 includes a shelf 114 that extends from the inside edge 123. In some examples, the shelf 114 extends in a direction A1. The shelf 114 may extend from the inside edge 123. The circuit substrate 110 contacts the shelf 114.

As shown in FIG. 1B, the shelf 114 may include one or more shelf portions (e.g., also referred to as shoulder portions) that extend from the inside edge 123 of the frame 140 in the directions A1 and A2. The shelf 114 may have a thickness that extends in the direction A3. In some examples, the shelf portions extend from the inside edge 123 in the direction A2 and/or the direction A3 at multiple different lengths. As shown in FIG. 1C, the shelf portions may include a corner portion 135, a corner portion 137, a corner portion 139, and a corner portion 141. Each of the corner portion 135, the corner portion 137, and the corner portion 139 may define, include, or contact a protrusion 116. The shelf portions may include a connecting portion 131 that extends between the corner portion 135 and the corner portion 137. The shelf portions may include a connecting portion 133 that extends between the corner portion 137 and the corner portion 139.

The circuit substrate 110 may sit on top of the shelf 114 such that the circuit substrate 110 is positioned within the frame 140. As shown in FIG. 1G, the circuit substrate 110 includes a first surface 151 and a second surface 153 that is opposite to the first surface 151. The first surface 151 is disposed in a plane A4. The thickness of the circuit substrate 110 is defined as the distance between the first surface 151 and the second surface 153 in the direction A3. The direction A3 is orthogonal to the plane A4. The direction A1 is perpendicular to the direction A3 and perpendicular to the direction A2. The direction A2 is perpendicular to the direction A1 and perpendicular to the direction A3. The circuit substrate 110 includes one or more holes 115a. In some examples, the holes 115a extend entirely through the thickness of the circuit substrate 110 in the direction A3. The protrusions 116 may extend through the holes 115a of the circuit substrate 110 and may extend into the holes 115a of the structural liner 145.

A structural liner 145 is a reinforcement member that provides reinforcement or support to one or more components included in the fluidic manifold 108 such as the circuit substrate 110. In some examples, the structural liner 145 includes a perimeter portion 171 that extends between the circuit substrate 110 and the inside edge 123 of the fluidic manifold. In some examples, the perimeter portion 171 may have a shape that corresponds to a shape of the inside edge 123. In some examples, the structural liner 145 includes an interior portion 173 that covers an interior area having one or more fluid components 174 and/or pressure sensors 186. In some examples, the structural liner 145 includes an opening that exposes at least a portion of the circuit substrate 110 to an inner surface of a first sidewall (e.g., first sidewall 232) or an inner surface of a second sidewall (e.g., second sidewall 234). The fluidic components 174 may include one or more pumps and one or more valves. The interior portion 173 of the structural liner 145 may define one or more openings that correspond to the fluidic components 174 and/or the pressure sensors 186.

In some examples, the structural liner 145 may include one or more circumferential portions 187. A circumferential portion 187 may extend from the perimeter portion 171 in a direction A2 and/or A1. A circumferential portion 187 may cover a portion of the circuit substrate 110. In some examples, a circumferential portion 187 is disposed over a respective corner portion (e.g., corner portion 135, corner portion 137, corner portion 139). A circumferential portion 187 may include a female feature such as a hole 115b, which may receive a protrusion 116 extending from the shelf 114 of the fluidic manifold 108 and extending through a hole 115a of the circuit substrate 110. In some examples, the circumferential portion 187 defines a male feature, and the fluidic manifold 108 defines a female feature.

The structural liner 145 is coupled to the fluidic manifold 108 using one or more coupling members 113. The holes 115b and the protrusions 116 are examples of one type of coupling member 113. In some examples, one or more portions of the circuit substrate 110 are disposed between the structural liner 145 and the fluidic manifold 108. In general, a coupling member 113 may be one of a variety of components that can couple the structural liner 145 to the fluidic manifold 108 to secure the circuit substrate 110 and other components of the fluidic manifold 108. A coupling member 113 may be two interacting or interlocking components such as a first interacting component on the fluidic manifold 108 and a second interacting component on the structural liner 145. In some examples, a coupling member 113 may include a male feature defined on the fluidic manifold 108 or on the structural liner 145 and a female feature defined on the structural liner 145 or on the fluidic manifold 108. In some examples, the male feature is a protrusion 116 (e.g., solid or threaded or patterned), and a female feature is a hole 115b, which can be coupled together using an interference fit, a clip, a ring, an adhesive material, material flow, etc.

In some examples, the structural liner 145 contacts the fluidic manifold 108 and is disposed around the circuit substrate 110. In some examples, the structural liner 145 contacts an edge 195 (e.g., see FIG. 1G) around the circuit substrate 110. In some examples, the perimeter portion 171 of the structural liner 145 contacts the edge 195. The edge 195 extends from the first surface 151 of the circuit substrate 110 and the second surface 153 of the circuit substrate 110 in the direction A3. The structural liner 145 may contact the shelf 114. In some examples, the perimeter portion 171 of the structural liner 145 may contact the shelf 114. In some examples, the structural liner 145 (e.g., the perimeter portion 171) may contact a portion 159 of the shelf 114 that is disposed between the edge 195 of the circuit substrate 110 and the inside edge 123 of the fluidic manifold 108 in the direction A1. In some examples, the structural liner 145 may contact the inside edge 123 of the fluidic manifold 108. In some examples, the structural liner 145 may contact the circuit substrate 110. In some examples, the structural liner 145 may contact portions of the first surface 151 of the circuit substrate 110. In some examples, the circumferential portion 187 may contact the first surface 151 of the circuit substrate 110.

As shown in FIG. 1A, the implantable medical device 100 includes a fluid reservoir 102, and an inflatable member 104. The electronic pump device 106 is configured to transfer fluid between the fluid reservoir 102 and the inflatable member 104. In some examples, the inflatable member 104 is an inflatable cuff member configured to be implemented around a urethra of a patient. In some examples, the inflatable member 104 is a penile inflation member (e.g., one or more inflatable cylinders) that may be implanted into the corpus cavernosum of the user. The fluid reservoir 102 may be implanted in the abdomen or pelvic cavity of the user (e.g., the fluid reservoir 102 may be implanted in the lower portion of the user’s abdominal cavity or the upper portion of the user’s pelvic cavity). In some examples, at least a portion of the electronic pump device 106 may be implemented in the patient’s body.

The inflatable member 104 may be capable of expanding upon the injection of fluid into a cavity of the inflatable member 104. If implanted around the urethra, the expansion of the inflatable member 104 causes the urethra to become restricted, thereby reducing the risk of incontinence in patients. For example, the electronic pump device 106 is configured to move fluid to pressure the inflatable cuff (e.g., the inflatable member 104), which constricts the urethra, thereby restricting the flow of urine. To urinate, the patient may operate the electronic pump device 106 to depressurize the inflatable cuff by transferring fluid from the inflatable cuff to the fluid reservoir 102. If implanted into the corpus cavernosum, upon injection of the fluid into the inflatable member 104, the inflatable member 104 may increase its length and/or width, as well as increase its rigidity.

The fluid reservoir 102 may include a container having an internal chamber configured to hold or house fluid that is used to inflate the inflatable member 104. In some examples, the fluid reservoir 102 is pressurized. In some examples, the fluid reservoir 102 is a pressurized balloon. In some examples, the implantable medical device 100 includes a single pressurized balloon. In some examples, the implantable medical device 100 includes two or more pressurized balloons. The pressure in the inflatable member 104 may be generated by the fluid reservoir 102.

The implantable medical device 100 may include a first tube member 103 and a second tube member 105. In some examples, the first tube member 103 and the second tube member 105 are referred to as conduit connectors. Each of the first tube member 103 and the second tube member 105 may define a lumen configured to transfer the fluid to and from the electronic pump device 106. The first tube member 103 may be coupled to the electronic pump device 106 and the fluid reservoir 102 such that fluid can be transferred between the electronic pump device 106 and the fluid reservoir 102 via the first tube member 103. For example, the first tube member 103 may define a first lumen configured to transfer fluid between the electronic pump device 106 and the fluid reservoir 102. The first tube member 103 may include a single or multiple tube members for transferring the fluid between the electronic pump device 106 and the fluid reservoir 102. In some examples, the first tube member 103 may be referred to as first tube members, and two first tube members can be connected together using a connector.

The second tube member 105 may be coupled to the electronic pump device 106 and the inflatable member 104 such that fluid can be transferred between the electronic pump device 106 and the inflatable member 104 via the second tube member 105. For example, the second tube member 105 may define a second lumen configured to transfer fluid between the electronic pump device 106 and the inflatable member 104. The second tube member 105 may include a single or multiple tube members for transferring the fluid between the electronic pump device 106 and the inflatable member 104. In some examples, the second tube member 105 may be referred to as second tube members, and two second tube members can be connected together using a connector. In some examples, the first tube member 103 and the second tube member 105 may include a silicone rubber material. In some examples, the electronic pump device 106 may be directly connected to the fluid reservoir 102.

The electronic pump device 106 that can monitor control and regulate the pressure within an inflatable member 104. In some examples, the electronic pump device 106 is referred to as a can. The electronic pump device 106 may automatically transfer fluid between the fluid reservoir 102 and the inflatable member 104 without the user manually operating a pump (e.g., squeezing and releasing a pump bulb). The electronic pump device 106 may include an antenna configured to wirelessly transmit (and receive) wireless signals from an external device 101. The external device 101 may be any type of component that can communicate with the electronic pump device 106. The external device 101 may be a computer, smartphone, tablet, pendant, key fob, etc. A user may use the external device 101 to control the implantable medical device 100. In some examples, the user may use the external device to inflate or deflate the inflatable member 104.

FIG. 2 illustrates an example of an exploded view of an electronic pump device 206 according to an aspect. The housing 220 includes a first sidewall 232, a second sidewall 234, a peripheral wall 236, and a frame 240. The first sidewall 232, second sidewall 234, and the peripheral wall 236 are hermetically sealed together to form an internal compartment 250 within the housing 220.

The frame 240 is disposed within the internal compartment 250 to form a first partition 252 and a second partition 254 in such a manner that the first partition 252 is hermetically sealed from the second partition 254. The frame 240 can be integrally formed with the peripheral wall 236, the first sidewall 232, and/or the second sidewall 234. In some examples, the frame 240 is welded to the peripheral wall 236 or welded to the first sidewall 232, and/or the second sidewall 234. The first sidewall 232, the peripheral wall 236, and the frame 240 may form the first partition 252. The second sidewall 234, the peripheral wall 236, and the frame 240 may form the second partition 254, which is opposite the frame 240 from the first partition 252.

The electronic pump device 206 can include a header 226 attached to the housing 220 to form an internal region 258 between an inner surface of the header 226 and an outer surface of the housing 220 that includes power and communication interface structures such as a secondary coil 228 and the antenna 230 external to the hermetically sealed housing 220. The header 226 is configured from a dielectric or insulative material, such as a radome, to allow the transmission of power and communication signals between the antenna 230 and a handset programmer or charger, and between the secondary coil 228 and the charger. For example, the header 226 may include an over-molded polymer affixed to the housing 220 and including the secondary coil 228 and the antenna 230 within the internal region 258. The secondary coil 228 and antenna 230 are constructed from a biocompatible material. In some examples, the secondary coil 228 and antenna 230 can be formed as a coil from a stamped titanium core clad with gold or silver. In some examples, the secondary coil 228 and antenna 230 can be formed from a gold wire.

The electronic pump device 206 includes an energy storage system, such as a battery (e.g., a rechargeable power source) (e.g., a rechargeable battery), and electronic components 212 within the first partition 252. The electronic components 212 can be disposed on a circuit substrate 210, such as a plurality of circuit boards, within the first partition 252. The battery 260 can assume various forms appropriate to provide power for generating desired electrical signals and to store power provided from the electronic components 212. For example, the battery 260 can incorporate lithium-ion (Li+) chemistry, e.g., a lithium-ion battery to operate the electronic components 212. In some examples, the electronic components 212 can be implemented by various components including resistors, capacitors, transistors, and integrated circuits disposed on the circuit substrate 210. The secondary coil 228 and antenna 230 are electrically coupled to the electronic components 212 within the first partition 252, such as via a hermetic feedthrough component.

The electronic components 212 can include a recharge system, a communication system, and a controller. The recharge system includes hardware configured to interface with the secondary coil 228 to receive power signals, and to provide the power signals in a form suitable to recharge the battery 260 and can include circuitry to reduce the likelihood of overcharging the battery 260. The communication system includes hardware configured to interface with the antenna 230 to receive electrical communication signals. For instance, the communication system can be configured to communicate via a wireless personal area network technology such as a short-range communication protocol (e.g., Bluetooth) (e.g., Bluetooth Low Energy), which is compatible with several operating systems that can be applied in mobile devices configured as external devices (e.g., external device 101) (e.g., handset programmers). The communication system can include an integrated circuit to implement an applied communication technology. In some examples, the communication system can be used to transmit communication signals to other devices, such as a charger or the handheld programmer (e.g., external device 101), and the communication system can be implemented to generate communication signals and provide the communication signals to the antenna 230 for transmission. In some examples, the communication system can be configured to receive and transmit radio frequency signals via the antenna 230. The controller can include a microcontroller to operate the recharge system and to receive and operate in response to communication signals or generate communication signals from the communication system.

The electronic pump device 206 also includes a fluidic circuit 270 within the second partition 254 and opposite the frame 240 from the battery 260 and electronic components 212. In some examples, the frame 240 can include an opening 242 that includes a hermetic interface 244, such as a feedthrough hermetically affixed to the frame 240. The electronic components 212 are operably coupled to the fluidic circuit 270 across the frame 240 via the hermetic interface 244. For example, the controller of the electronic components 212, powered by the battery 260, can cause the operation of the fluidic circuit 270 such as to control and monitor the fluidic circuit 270.

The fluidic circuit 270 includes a fluidic manifold 208 and fluidic components 274 operably coupled to the fluidic manifold 208. In some examples, the fluidic manifold 208 is a structure integrated into the frame 240 such that the fluidic manifold 208 and the frame 240 together form the hermetic barrier between the first partition 252 and the second partition 254 of the internal compartment 250. For instance, the battery 260, the circuit substrate 210, or electronic components 212 can be coupled to a first major surface of the fluidic manifold 208 in the first partition 252, and the fluidic components 274 are operably coupled to a second, and opposite major surface of the fluidic manifold 208 in the second partition 254.

The fluidic circuit 270 provides for the transfer of the fluid between the fluid reservoir (e.g., the fluid reservoir 102 of FIG. 1A) and the inflatable member (e.g., the inflatable member 104 of FIG. 1A). The fluidic manifold 208, which can be a hermetic manifold, segments and contains the fluid from the internal compartment 250 to reduce the chance of fluid exchange and directs the fluid from a first port 276 to a second port 278 via internal fluid passageways or channels.

The fluidic components 274 include a plurality of fluid pumps, such as pumps 280, 282, a valve 284 mounted into the fluidic manifold 208 in fluidic communication with a manifold passageway to transfer fluid from the first port 276 to the second port 278. The pumps and the valve(s) are in fluid communication with a single fluid passageway between ports 276, 278. The fluidic components 274 also includes one or more pressure sensors 286 operably coupled to the fluidic manifold 208 and in fluidic communication with the passageway to detect a pressure of the fluid within the fluidic manifold 208.

The fluidic components 274 are included in a planar configuration on the fluidic manifold 208 in which the pumps 280, 282, valve 284, and pressure sensor 286 are mounted into the fluidic manifold 208 on a plane for slim profile within the second partition 254. The fluidic manifold 208 can include chambers 288 formed into the second major surface in which the chambers are fluidically coupled to the single passageway within the fluidic manifold 208. The chambers are configured to receive the pumps 280, 282, and valve 284 and one or more pressure sensors 286. In some examples, the fluidic manifold 208 can receive a piezoelectric pump. The fluidic manifold 208 can receive a component cover 290 over the fluidic components 274, which can be hermetically sealed to the second major surface.

In some examples, the electronic pump device 206 may include kink resistant tubing 292 that can extend through the header 226 and attached to the ports 276, 278 via components such as a barb 294 and O-rings. The kink resistant tubing 292 can be attached to the tube members 103, 105 of FIG. 1A to fluidically couple the electronic pump device 206 to the fluid reservoir (e.g., the fluid reservoir 102 of FIG. 1A) and the inflatable member (e.g., the inflatable member 104 of FIG. 1A).

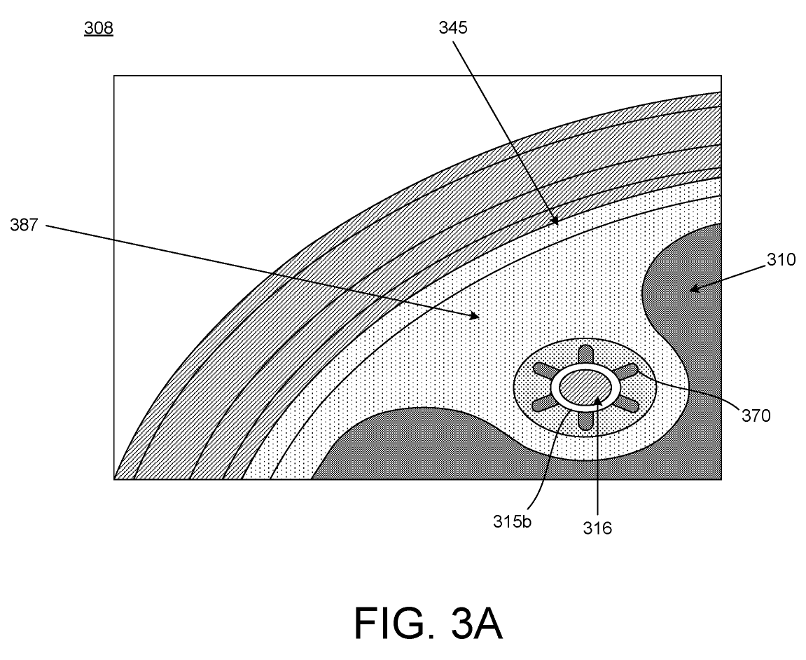

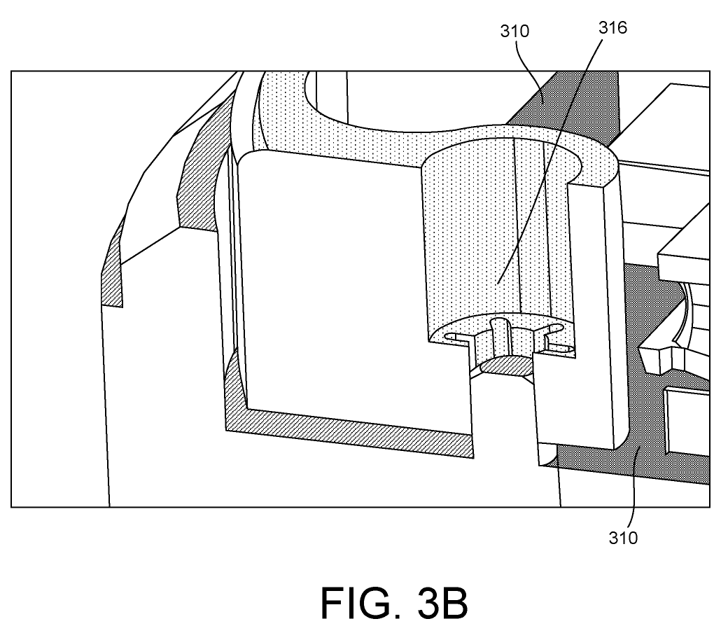

FIGS. 3A and 3B illustrate a portion of a fluidic manifold 308 with a structural liner 345 and a circuit substrate 310 that are coupled to the fluidic manifold 308 based on an interference fit. FIG. 3A illustrates a circumferential portion 387 that allows for the structural liner 345 to be displaced while being assembled but provides some compression on a protrusion 316 to remain in place after assembly. In some examples, the area between grooves 370 of the patterned geometry can be backfilled with adhesive to provide further affixation. FIG. 3B illustrates a cross-sectional view of the fluidic manifold of FIG. 3A.

Referring to FIGS. 3A and 3B, the structural liner 345 may be coupled to the fluidic manifold 308 based on a press-fit or interference fit. The fluidic manifold 308 includes a male feature, such as a protrusion 316. In some examples, the protrusion 316 includes a post (e.g., a cylindrical post). The structural liner 345 includes a hole 315b configured to receive the protrusion 316. In some examples, the structural liner 345 includes grooves 370 that extend radially from the hole 315b. In some examples, the hole 315b has a size (e.g., a diameter) that is less than a size (e.g., a diameter) of the protrusion 316, thereby providing an interference fit to minimize or prevent movement. In some examples, an assembly operator may forcibly press-fit the structural liner 345 onto the protrusions 316 (e.g., manifold posts) in one, two, or more locations, thereby securing the circuit substrate 310 and minimizing (e.g., preventing) movement of the device’s component relative to one another.

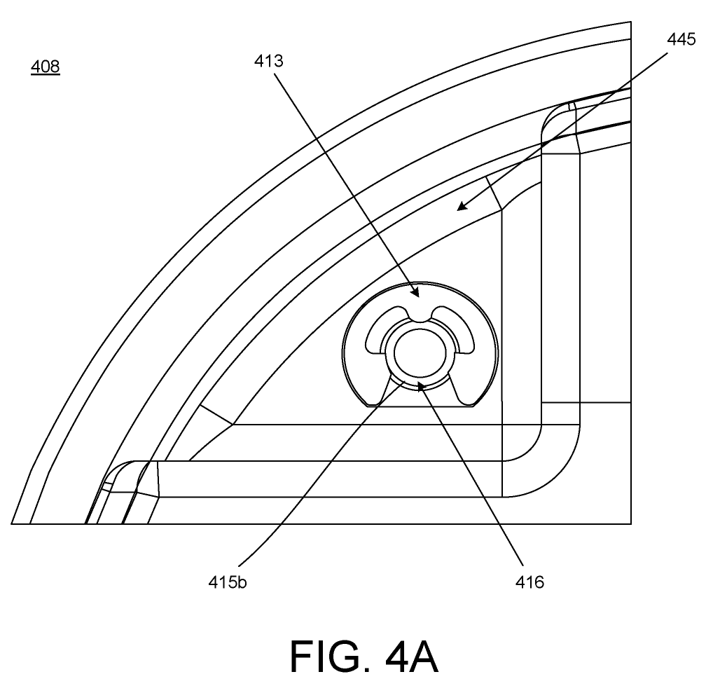

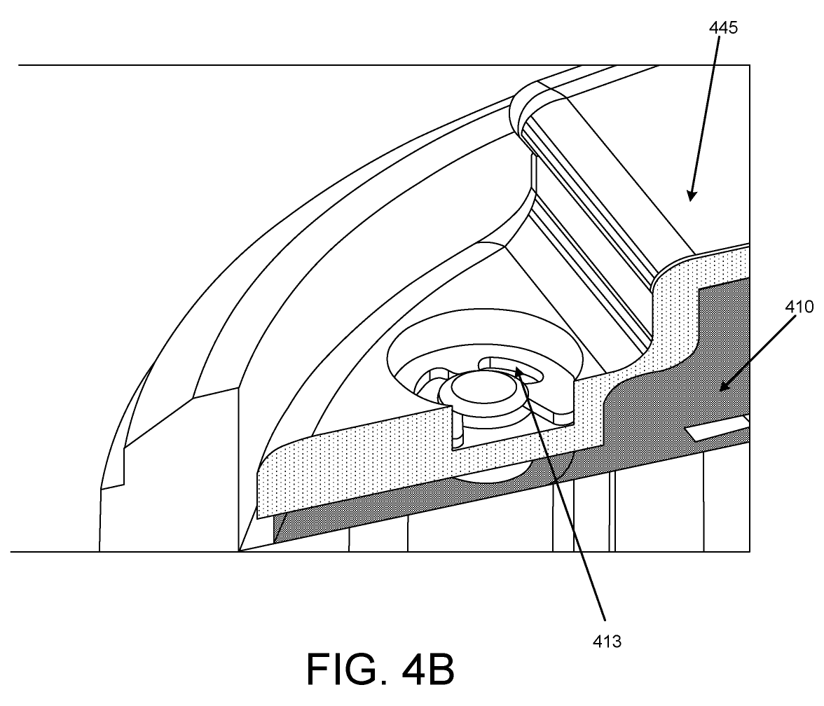

FIGS. 4A and 4B illustrate a portion of a fluidic manifold 408 with a structural liner 445 and a circuit substrate 410 that are coupled to the fluidic manifold 408 using one or more clips 413. In some examples, a clip 413 includes a ring with a slot. In some examples, a clip 413 includes prongs (e.g., three prongs) that contact a protrusion 416. The slot may have a size less than a size of the protrusion 416. In some examples, the protrusion 416 includes a cylindrical post. In some examples, the protrusion 416 is a solid (e.g., smooth) post. In some examples, the protrusion 416 includes a post with one or more groove cuts in which the clip 413 sits into. The structural liner 445 includes a hole 415b configured to receive the protrusion 416, where the protrusion 416 extends through the other side of the structural liner 445, and the clip 413 is attached to the protrusion 416. In some examples, the clips 413 are e-style or c-style clips. The clips 413 can be pushed on the male features (e.g., the protrusions 416), thereby mechanically locking the structural liner 445 and a circuit substrate 410 to the fluidic manifold 108 (e.g., the manifold’s shelf). For example, a clip 413 can be pushed onto a protrusion 416 to hold the structural liner 445 and the circuit substrate 410.

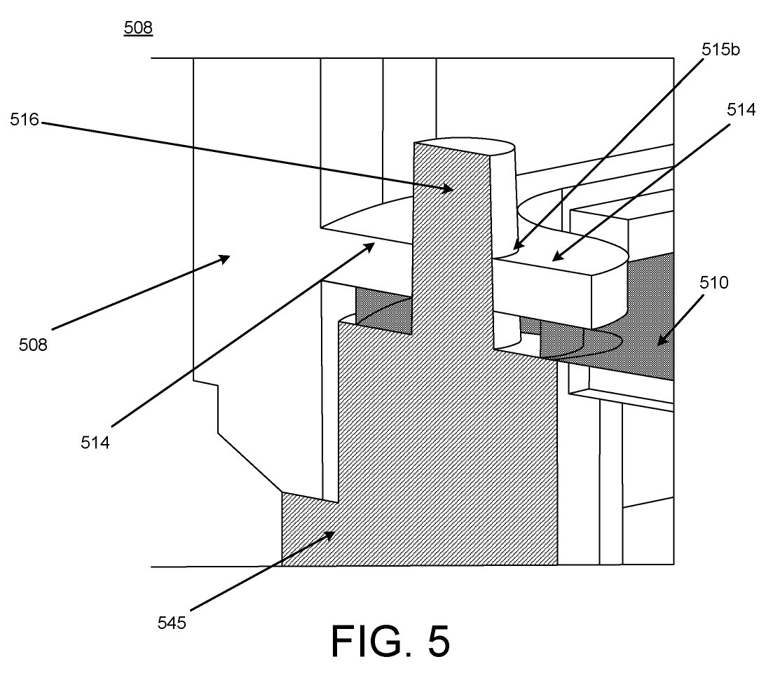

FIG. 5 illustrates a portion of a fluidic manifold 508 with a structural liner 545 and a circuit substrate 510 that are coupled to the fluidic manifold 508 based on an interference fit. Referring to FIG. 5, the structural liner 545 includes a protrusion 516 (e.g., a male feature) and a shelf 514 of the fluidic manifold 508 includes a hole 515b (e.g., a female feature). Referring to FIG. 5, the structural liner 445 may be coupled to the fluidic manifold 508 based on a press-fit or interference fit. In some examples, the protrusion 516 includes a post (e.g., a cylindrical post). In some examples, the hole 515b has a size (e.g., a diameter) that is less than a size (e.g., a diameter) of the protrusion 516, thereby providing an interference fit to minimize or prevent movement.

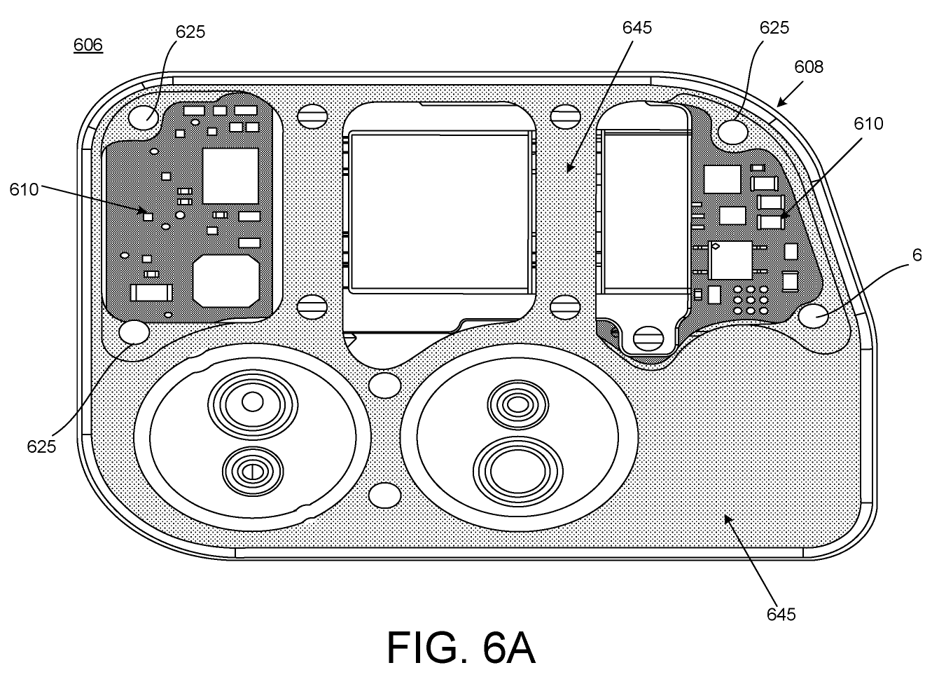

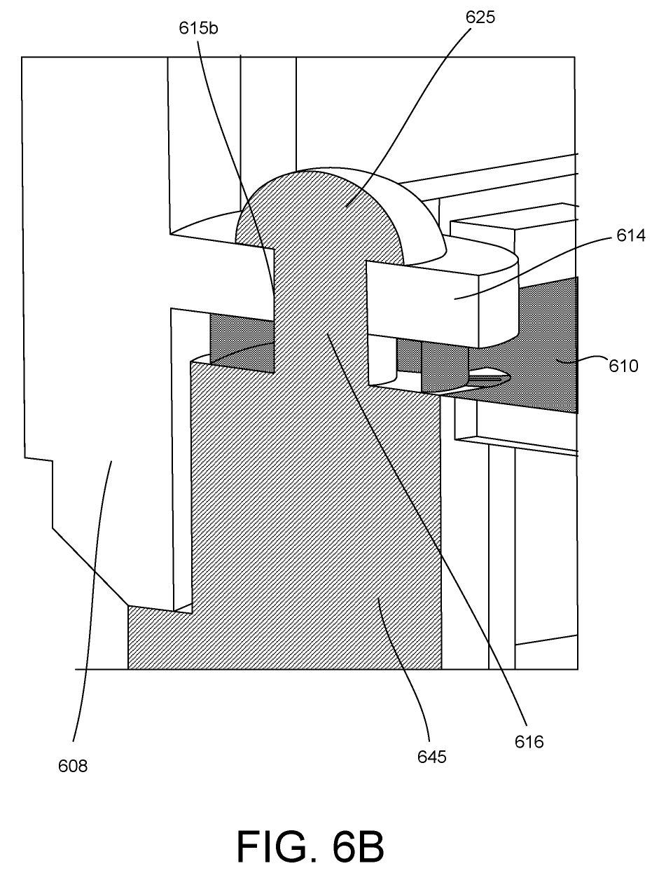

FIGS. 6A and 6B illustrate a portion of an electronic pump device 606 with a fluidic manifold 608 having a structural liner 645 and a circuit substrate 610 that are coupled to the fluidic manifold 608 based on a heat stake or material reflow retainment. For example, a portion of a protrusion 616 may be heated (e.g., melted) to create an enlarged end portion 625 (e.g., a mechanical lock feature), thereby securing the circuit substrate 610 and the structural liner 645 to a shelf 614 of the fluidic manifold 608.

Referring to FIGS. 6A and 6B (and similar to FIG. 5), the structural liner 645 includes a protrusion 616 (e.g., a male feature). The shelf 614 of the fluidic manifold 608 includes a hole 615b (e.g., a female feature). Referring to FIGS. 6A and 6B, the structural liner 645 may be coupled to the fluidic manifold 608 by inserting the protrusion 616 into the hole 615b, and heating the end portion of the protrusion 616, to create the enlarged end portion 625. In some examples, the hole 615b has a size (e.g., a diameter) that is less than a size (e.g., a diameter) of the protrusion 616, thereby providing an interference fit to minimize or prevent movement.

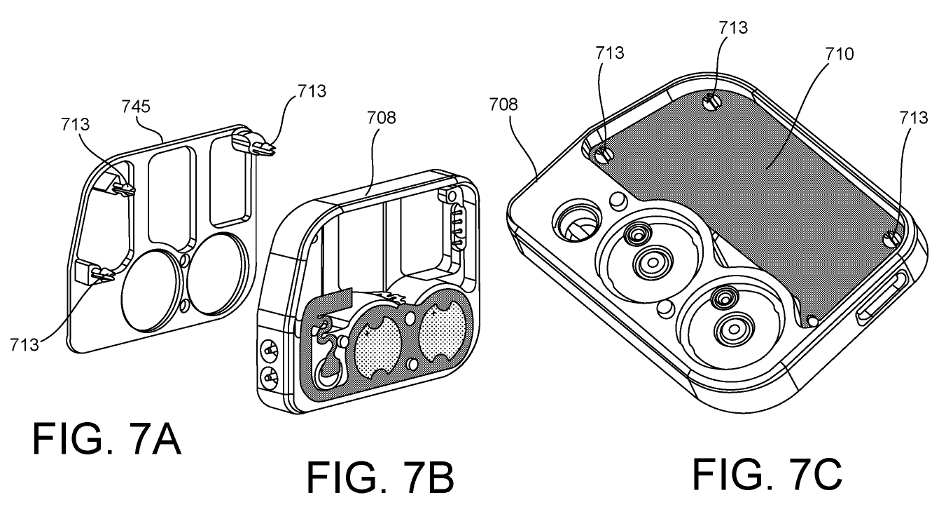

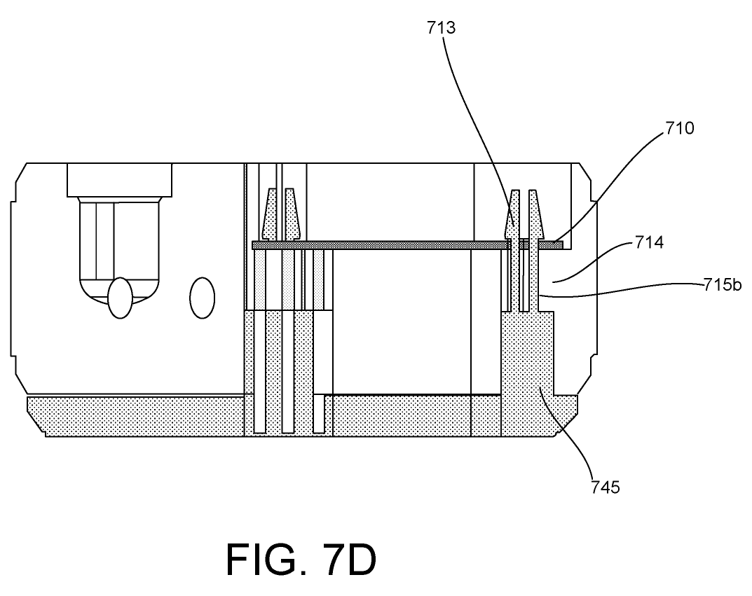

FIGS. 7A to 7D illustrate a portion of a fluidic manifold 708 with a structural liner 745 and a circuit substrate 710 that are coupled to the fluidic manifold 708 using one or more rivets 713. The structural liner 745 includes rivets 713. In some examples, a rivet 713 is integral to the base of the structural liner 745. The fluidic manifold 708 includes a shelf 714 defining a hole 715b that receives a rivet 713. In some examples, a rivet 713 is a blind rivet with a countersunk head.

The rivet 713 can be assembled from either direction, top or bottom, and may be deployed through the hole in the circuit substrate 710 and the hole 715b in a shelf 714 of the fluidic manifold 708 to constrain both the circuit substrate 710 and the structural liner 745 to the fluidic manifold 708. The deployed rivet 713 grabs the bottom of the hole 715b in the fluidic manifold 708 from the top or the top of the circuit substrate 10 from the bottom, thereby locking the circuit substrate 710 in place. In some examples, the rivets 713 could also extend further and mate with a similar female feature(s) on a top liner that would sandwich the fluidic manifold 708 and the circuit substrate 710 between both liners and hold the subassembly together.

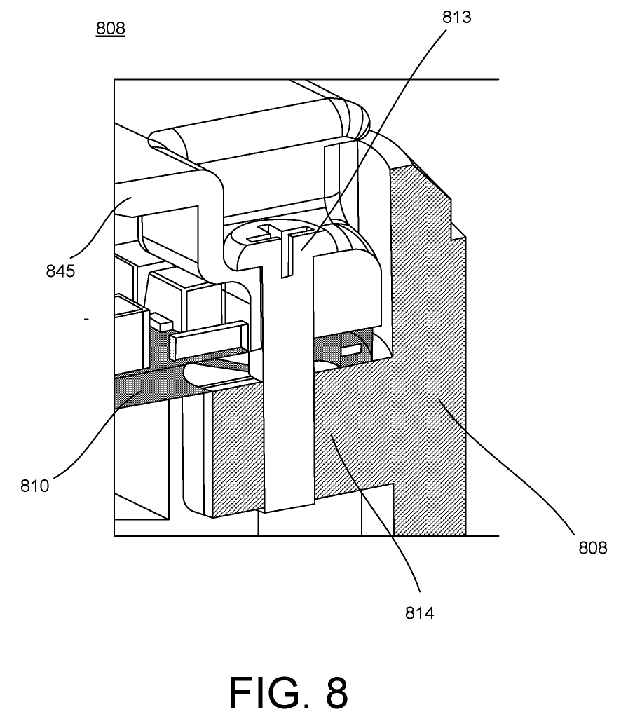

FIG. 8 illustrates a portion of a fluidic manifold 808 having a structural liner 845 and a circuit substrate 810 that are coupled to the fluidic manifold 808 using one or more fasteners 813. The structural liner 845 may define a first hole, the circuit substrate 810 may define a second hole, and a shelf 814 of the fluidic manifold 808 may define a third hole. The first to third holes may be aligned, and a fastener 813 may be inserted into the holes. In some examples, the fastener 813 and/or holes may be threaded. In some examples, the fastener 813 is inserted from the top side of the structural liner 845. The holes of the structural liner 845 and the circuit substrate 810 may be slightly larger than the threaded diameter allowing the fastener 813 to pass through them and engage with the threads on the fluidic manifold 808.

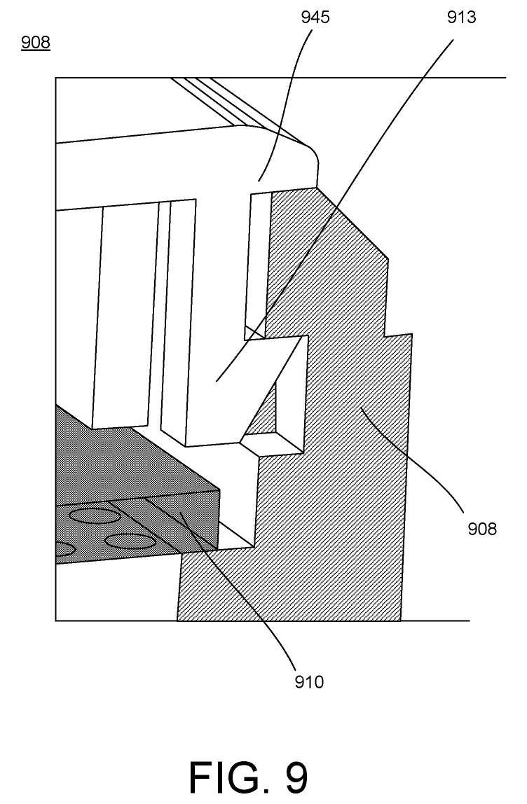

FIG. 9 illustrates a cross-sectional view of a portion of a fluidic manifold 908 with a female undercut and a structural liner 945 with a snap-fit feature 913. FIG. 9 illustrates another method for conjoining a structural liner 945 and a fluidic manifold 908 (and, in some examples, a circuit substrate 910) by using an undercut feature (e.g., a female uncut) on the fluidic manifold 908 to engage with a snap fit feature 913 on the structural liner 945. The snap-fit feature 913 includes a protruding flexible element.

The protruding flexible element can be pushed past a rigid element on the mating part (e.g., the undercut feature). Once past the rigid element, the protruding flexible element may snap back into its original shape, thereby creating a secure hold. The undercut feature may define a lip, an inward-facing slop, an L-shaped feature, or a dovetail feature (e.g., a wider undercut with angled sides). The undercut feature may operate as a retaining member that may minimize or prevent the snap-fit feature 913 from sliding back out. In some examples, the undercut feature is defined by a cutout portion in the inside edge of the fluidic manifold 908. Although one cantilever snap-fit joint is depicted, two or more snap-fit features 913 can be used along the manifold frame for increased connectivity. The examples are not limited to this type of snap fit, where any type of joint may be used.

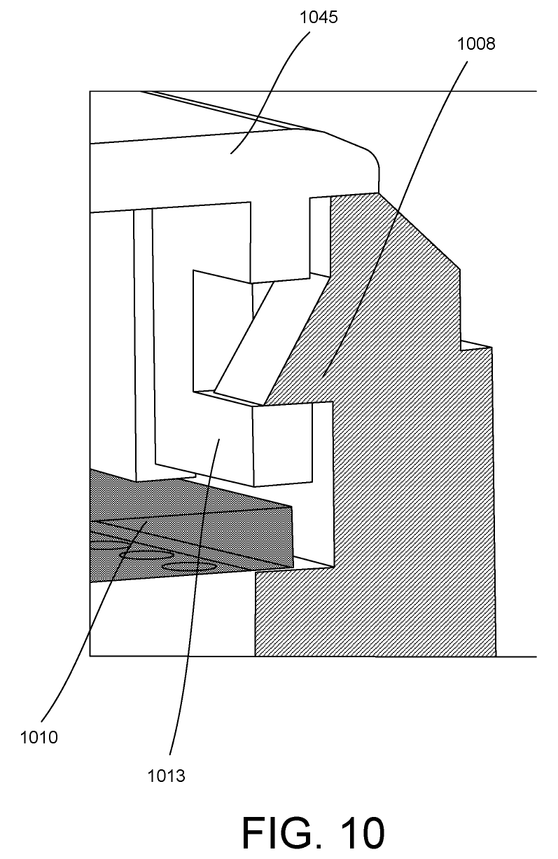

FIG. 10 illustrates a cross-sectional view of a portion of a fluidic manifold 1008 with a male undercut and a structural liner 1045 with a snap-fit feature 1013. FIG. 10 illustrates another method for conjoining a structural liner 1045 and a fluidic manifold 1008 (and, in some examples, a circuit substrate 1010) by using an undercut feature (e.g., a female uncut) on the fluidic manifold 1008 to engage with a snap fit feature 1013 on the structural liner 1045. The undercut feature on the fluidic manifold 1008 includes a protrusion (e.g., a sloped protrusion) that extends in the direction A1. The snap-fit feature includes a slot defining a flexible arm portion. The flexible arm portion may bend, thereby allowing the snap-fit feature 1013 to be inserted into the slot, thereby providing a secure connection. Although one cantilever snap-fit joint is depicted, two or more snap-fit features 1013 can be used along the manifold frame for increased connectivity. The examples are not limited to this type of snap fit, where any type of joint may be used.

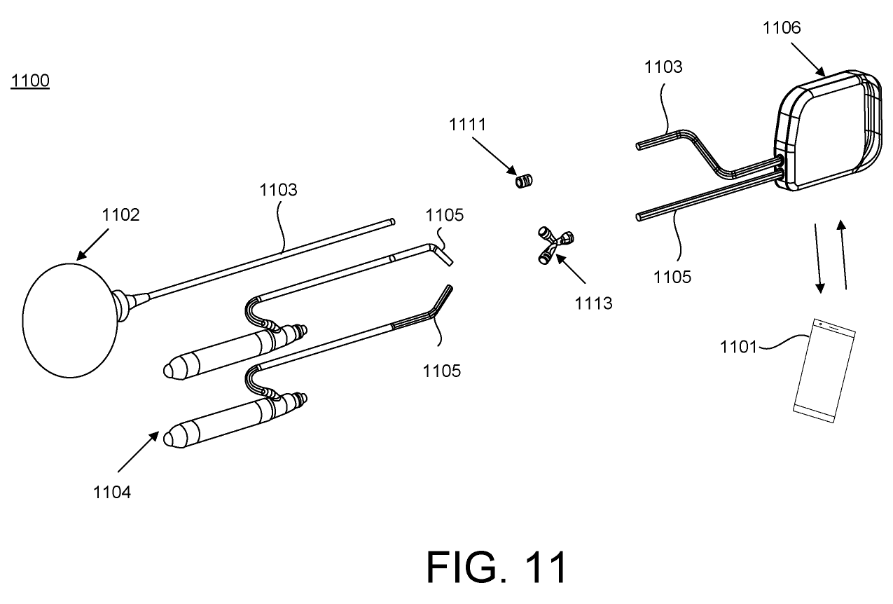

FIG. 11 illustrates a perspective of an inflatable penile prosthesis 1100 according to an aspect. The inflatable penile prosthesis 1100 may be an example of any of the medical devices discussed herein (e.g., including implantable medical device 100), and, therefore, may include any of the details discussed with reference to the previous figures.

The inflatable penile prosthesis 1100 includes an inflatable member 1104, a fluid reservoir 1102, and an electronic pump device 1106. The inflatable member 1104 includes a pair of inflatable cylinders. The electronic pump device 1106 may be an example of any of the pump devices discussed with reference to the previous figures and may include any of the details discussed herein. The electronic pump device 1106 includes fluidic components such as pumps, valves, and/or sensing devices positioned in fluid passageways. The electronic pump device 1106 includes components such as, for example, one or more fluid control devices, one or more pressure sensors, and other such components. The electronic pump device 1106 includes an electronic control system configured to provide for the transfer of fluid between a fluid reservoir 1102 and an inflatable member 1104 via the fluidic components.

The electronic pump device 1106 may include one or more integrated circuits. In some examples, the integrated circuits are included in a printed circuit board that is included in a housing of the electronic pump device 1106. Fluidic components and the electronic components of the electronic pump device 1106 are included in a housing. In some examples, fluidic components and electronic components in the housing define a manifold (e.g., an electronically controlled fluidic manifold) that provides for the electronic control of the flow of fluid between the fluid reservoir 1102 and the inflatable member 1104. In some examples, the electronic pump device 1106 can communicate with an external device 1101, via respective communication modules. For example, an application stored in a memory and executed by a processor of the external device 1101 may allow the user and/or a physician to operate, view, monitor and alter operation of the inflatable penile prosthesis 1100.

The inflatable penile prosthesis 1100 includes one or more first tube members 1103 that connect a first fluid port of the electronic pump device 1106 with the fluid reservoir 1102. One or more second tube members 1105 connect a second fluid port of the electronic pump device 1106 with the inflatable member 1104 in the form of the inflatable cylinders. In some examples, the inflatable penile prosthesis 1100 includes a connector 1111 that is used to connect two tube members 1103 together, and a connector 1113 that is used to connect two tube members 1105 together.

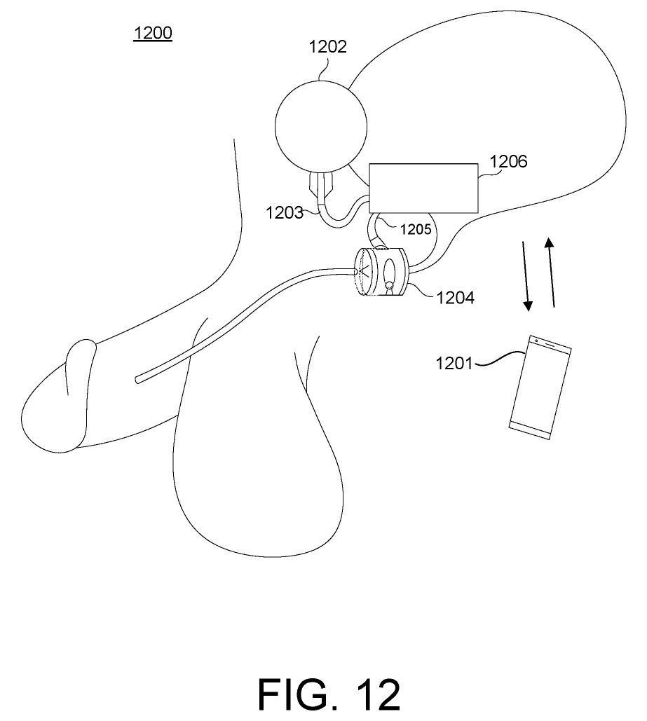

FIG. 12 illustrates a urinary control device 1200 having an electronic pump device 1206 according to an aspect. The urinary control device 1200 may be an example of the implantable medical device 100. In some examples, the urinary control device 1200 is an artificial urinary sphincter device. The electronic pump device 1206 may include any of the features of the pump devices discussed herein. The urinary control device 1200 includes an electronic pump device 1206, a fluid reservoir 1202, and a cuff 1204 (e.g., an inflatable cuff).

The fluid reservoir 1202 may be a pressure-regulating inflation balloon or element. The fluid reservoir 1202 is in operative fluid communication with the cuff 1204 via one or more tube members 1203, 1205. The fluid reservoir 1202 is constructed of polymer material that is capable of elastic deformation to reduce fluid volume within the fluid reservoir 1202 and push fluid out of the fluid reservoir 1202 and into the cuff 1204. However, the material of the fluid reservoir 1202 can be biased or include a shape memory construct adapted to generally maintain the fluid reservoir 1202 in its expanded state with a relatively constant fluid volume and pressure. In some examples, this constant level of pressure exerted from the fluid reservoir 1202 to the cuff 1204 will keep the cuff 1204 at a desired inflated state when open fluid communication is provided between the fluid reservoir 1202 and the cuff 1204. In some examples, the fluid reservoir 1202 is implanted into the abdominal space.

A user may use an external device 1201 to control the urinary control device 1200. In some examples, the user may use the external device 1201 to inflate or deflate the cuff 1204. For example, in response to the user activating an inflation cycle using the external device 1201, the external device 1201 may transmit a wireless signal to the electronic pump device 1206 to initiate the inflation cycle to transfer fluid from the fluid reservoir 1202 to the cuff 1204 (e.g., by opening an active valve where the pressure in the fluid reservoir 1202 causes the fluid to move through the active valve to the cuff 1204). In some examples, in response to the user activating a deflation cycle using the external device 1201, the external device 1201 may transmit a wireless signal to the pump device 1206 to initiate the deflation cycle to transfer fluid from the cuff 1204 to the fluid reservoir 1202.

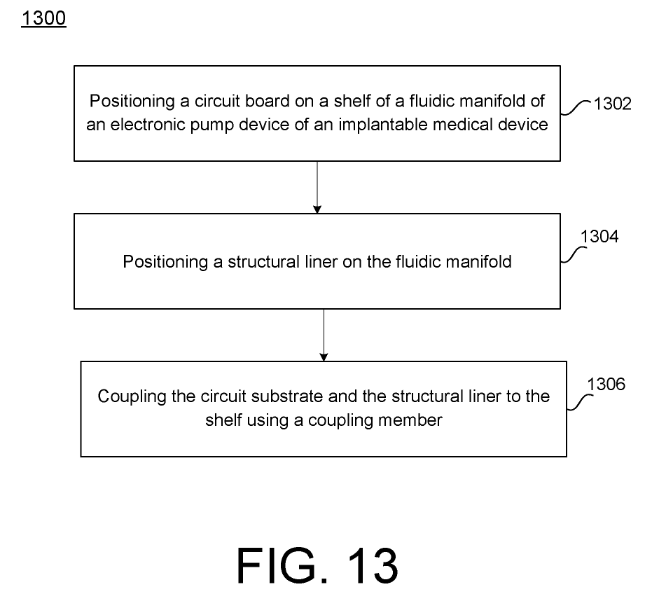

FIG. 13 illustrates a flowchart 1300 depicting example operations of coupling a circuit substrate and a structural liner to a fluidic manifold of an electronic pump device according to an aspect. Although the flowchart 1300 of FIG. 13 illustrates the operations in sequential order, it will be appreciated that this is merely an example, and that additional or alternative operations may be included. Further, operations of FIG. 13 and related operations may be executed in a different order than that shown, or in a parallel or overlapping fashion.

Operation 1302 includes positioning a circuit substrate on a shelf of a fluidic manifold of an electronic pump device of an implantable medical device. Operation 1304 includes positioning a structural liner on the fluidic manifold. Operation 1306 includes coupling the circuit substrate and the structural liner to the shelf using a coupling member.

Clause 1. An implantable medical device comprising: an inflatable member; a fluid reservoir; and an electronic pump device configured to transfer fluid between the inflatable member and the fluid reservoir, the electronic pump device including a housing, the housing including: a fluidic manifold including a plurality of fluidic components, the fluidic manifold including an inside edge and a shelf that extends from the inside edge; a circuit substrate contacting the shelf; and a structural liner connecting the inside edge and the shelf.

Clause 2. The implantable medical device of clause 1, wherein the fluidic manifold includes a protrusion that extends from the shelf, the circuit substrate including a first hole, the structural liner including a second hole, the protrusion extending through the first hole and the second hole.

Clause 3. The implantable medical device of clause 2, wherein the fluidic manifold includes a clip coupled to the protrusion.

Clause 4. The implantable medical device of clause 2, wherein the protrusion includes an enlarged end portion formed by heating the protrusion.

Clause 5. The implantable medical device of clause 1, wherein the structural liner includes a protrusion, the circuit substrate including a first hole, the shelf including a second hole, the protrusion extending through the first hole and the second hole.

Clause 6. The implantable medical device of clause 5, wherein the protrusion includes a rivet.

Clause 7. The implantable medical device of clause 1, wherein the structural liner includes a first hole, the circuit substrate includes a second hole, and the shelf includes a third hole, the fluidic manifold including a fastener that extends through the first hole, the second hole, and the third hole.

Clause 8. An electronic pump device for an inflatable medical device, the electronic pump device comprising: a fluidic manifold including a plurality of fluidic components, the fluidic manifold including an inside edge and a shelf that extends from the inside edge; a circuit substrate contacting the shelf; and a structural liner connecting the inside edge and the shelf.

Clause 9. The electronic pump device of clause 8, wherein the fluidic manifold includes a protrusion that extends from the shelf, the circuit substrate including a first hole, the structural liner including a second hole, the protrusion extending through the first hole and the second hole.

Clause 10. The electronic pump device of clause 9, wherein the fluidic manifold includes a clip coupled to the protrusion.

Clause 11. The electronic pump device of clause 9, wherein the protrusion includes an enlarged end portion formed by heating the protrusion.

Clause 12. The electronic pump device of clause 8, wherein the structural liner includes a protrusion, the circuit substrate including a first hole, the shelf including a second hole, the protrusion extending through the first hole and the second hole.

Clause 12a. The electronic pump device of any one of clauses 8 to 12, wherein the fluidic manifold includes a first sidewall, a second sidewall, a peripheral wall, and a frame, wherein the first sidewall, the peripheral wall, and the frame form a first partition, wherein the second sidewall, the peripheral wall, and the frame form a second partition, the first partition including the circuit substrate, the second partition including the plurality of fluidic components.

Clause 13. A method comprising: positioning a circuit substrate on a shelf of a fluidic manifold of an electronic pump device of an implantable medical device; positioning a

structural liner on the fluidic manifold; and coupling the circuit substrate and the structural liner to the shelf using a coupling member.

Clause 14. The method of clause 13, wherein the coupling member includes a protrusion that is integral to and extends from the shelf, the coupling member including a first hole defined on the circuit substrate and a second hole defined on the structural liner.

Clause 15. The method of clause 13, wherein the coupling member includes a protrusion that is integral to and extends from the structural liner, the coupling member including a first hole defined on the circuit substrate and a second hole defined on the shelf.

Clause 16. An implantable medical device comprising: an inflatable member; a fluid reservoir; and an electronic pump device configured to transfer fluid between the inflatable member and the fluid reservoir, the electronic pump device including a housing, the housing including: a fluidic manifold including a plurality of fluidic components, the fluidic manifold including an inside edge and a shelf that extends from the inside edge; a circuit substrate contacting the shelf; and a structural liner connecting the inside edge and the shelf.

Clause 17. The implantable medical device of clause 16, wherein the fluidic manifold includes a protrusion that extends from the shelf, the circuit substrate including a first hole, the structural liner including a second hole, the protrusion extending through the first hole and the second hole.

Clause 18. The implantable medical device of clause 17, wherein the fluidic manifold includes a clip coupled to the protrusion.

Clause 19. The implantable medical device of clause 17, wherein the protrusion includes an enlarged end portion formed by heating the protrusion.

Clause 20. The implantable medical device of clause 16, wherein the structural liner includes a protrusion, the circuit substrate including a first hole, the shelf including a second hole, the protrusion extending through the first hole and the second hole.

Clause 21. The implantable medical device of clause 20, wherein the protrusion includes a rivet.

Clause 22. The implantable medical device of clause 16, wherein the structural liner includes a first hole, the circuit substrate includes a second hole, and the shelf includes a third hole, the fluidic manifold including a fastener that extends through the first hole, the second hole, and the third hole.

Clause 23. The implantable medical device of clause 16, wherein the structural liner includes a snap-fit feature, and the shelf includes an undercut.

Clause 24. The implantable medical device of clause 23, wherein the undercut includes a female undercut.

Clause 25. The implantable medical device of clause 23, wherein the undercut includes a male undercut.

Clause 26. A method comprising: positioning a circuit substrate on a shelf of a fluidic manifold of an electronic pump device of an implantable medical device; positioning a structural liner on the fluidic manifold; and coupling the circuit substrate and the structural liner to the shelf using a coupling member.

Clause 27. The method of clause 26, wherein the coupling member includes a protrusion that is integral to and extends from the shelf, the coupling member including a first hole defined on the circuit substrate and a second hole defined on the structural liner.

Clause 28. The method of clause 26, wherein the coupling member includes a rivet.

Clause 29. The method of clause 26, wherein the coupling member includes a threaded fastener.

Clause 30. The method of clause 26, wherein the coupling member includes a protrusion that is integral to and extends from the structural liner, the coupling member including a first hole defined on the circuit substrate and a second hole defined on the shelf.

Clause 31. An electronic pump device for an inflatable medical device, the electronic pump device comprising: a fluidic manifold including a plurality of fluidic components, the fluidic manifold including an inside edge and a shelf that extends from the inside edge; a circuit substrate contacting the shelf; and a structural liner connecting the inside edge and the shelf.

Clause 32. The electronic pump device of clause 31, wherein the fluidic manifold includes a protrusion that extends from the shelf, the circuit substrate including a first hole, the structural liner including a second hole, the protrusion extending through the first hole and the second hole.

Clause 33. The electronic pump device of clause 32, wherein the fluidic manifold includes a clip coupled to the protrusion.

Clause 34. The electronic pump device of clause 32, wherein the protrusion includes an enlarged end portion formed by heating the protrusion.

Clause 35. The electronic pump device of clause 31, wherein the structural liner includes a protrusion, the circuit substrate including a first hole, the shelf including a second hole, the protrusion extending through the first hole and the second hole.

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implantable medical device comprising:
an inflatable member;
a fluid reservoir; and
an electronic pump device configured to transfer fluid between the inflatable member and the fluid reservoir, the electronic pump device including a housing, the housing including:
a fluidic manifold including a plurality of fluidic components, the fluidic manifold including an inside edge and a shelf that extends from the inside edge;
a circuit substrate contacting the shelf; and
a structural liner connecting the inside edge and the shelf.

2. The implantable medical device of claim 1, wherein the fluidic manifold includes a protrusion that extends from the shelf, the circuit substrate including a first hole, the structural liner including a second hole, the protrusion extending through the first hole and the second hole.

3. The implantable medical device of claim 2, wherein the fluidic manifold includes a clip coupled to the protrusion.

4. The implantable medical device of claim 2, wherein the protrusion includes an enlarged end portion formed by heating the protrusion.

5. The implantable medical device of claim 1, wherein the structural liner includes a protrusion, the circuit substrate including a first hole, the shelf including a second hole, the protrusion extending through the first hole and the second hole.

6. The implantable medical device of claim 5, wherein the protrusion includes a rivet.

7. The implantable medical device of claim 1, wherein the structural liner includes a first hole, the circuit substrate includes a second hole, and the shelf includes a third hole, the fluidic manifold including a fastener that extends through the first hole, the second hole, and the third hole.

8. The implantable medical device of claim 1, wherein the structural liner includes a snap-fit feature, and the shelf includes an undercut.

9. The implantable medical device of claim 8, wherein the undercut includes a female undercut.

10. The implantable medical device of claim 8, wherein the undercut includes a male undercut.

11. A method comprising:
positioning a circuit substrate on a shelf of a fluidic manifold of an electronic pump device of an implantable medical device;
positioning a structural liner on the fluidic manifold; and
coupling the circuit substrate and the structural liner to the shelf using a coupling member.

12. The method of claim 11, wherein the coupling member includes a protrusion that is integral to and extends from the shelf, the coupling member including a first hole defined on the circuit substrate and a second hole defined on the structural liner.

13. The method of claim 11, wherein the coupling member includes a rivet.

14. The method of claim 11, wherein the coupling member includes a threaded fastener.

15. The method of claim 11, wherein the coupling member includes a protrusion that is integral to and extends from the structural liner, the coupling member including a first hole defined on the circuit substrate and a second hole defined on the shelf.

16. An electronic pump device for an inflatable medical device, the electronic pump device comprising:
a fluidic manifold including a plurality of fluidic components, the fluidic manifold including an inside edge and a shelf that extends from the inside edge;
a circuit substrate contacting the shelf; and
a structural liner connecting the inside edge and the shelf.

17. The electronic pump device of claim 16, wherein the fluidic manifold includes a protrusion that extends from the shelf, the circuit substrate including a first hole, the structural liner including a second hole, the protrusion extending through the first hole and the second hole.

18. The electronic pump device of claim 17, wherein the fluidic manifold includes a clip coupled to the protrusion.

19. The electronic pump device of claim 17, wherein the protrusion includes an enlarged end portion formed by heating the protrusion.

20. The electronic pump device of claim 16, wherein the structural liner includes a protrusion, the circuit substrate including a first hole, the shelf including a second hole, the protrusion extending through the first hole and the second hole.
